(19) United States
(12) Patent Application Publication
(10) Pub. No.: US 2010/0136109 A1
(43) Pub. Date: Jun. 3, 2010

(54) SUSTAINED RELEASE

(76) Inventors: Richard Ross, Sheffield (GB); Hiep Huatan, Kent (GB)

Correspondence Address: KLARQUIST SPARKMAN, LLP, 121 SW SALMON STREET, SUITE 1600, PORTLAND, OR, 97204, US

(21) Appl. No.: 12/597,153

(22) PCT Filed: Apr. 22, 2008
(86) PCT No.: PCT/GB2008/001433

§ 371 Date: Oct. 23, 2009

(30) Foreign Application Priority Data
Apr. 23, 2007 (GB) 0707755.5
Nov. 27, 2007 (GB) 0723191.3

(51) Int. Cl. A61K 9/52; A61K 31/195; A61P 5/14

(52) U.S. Cl. 424/457; 514/567

(57) ABSTRACT

We describe a medicament for the treatment of thyroid disorders that typically result from a hypoactive thyroid gland that releases thyroxine and triiodothyronine in a sustained pattern when administered to a subject.

The invention relates to a medicament for the treatment of thyroid disorders that typically result from a hypoactive thyroid gland.

The delivery of drugs as oral preparations in combination with a delivery vehicle that confers controlled release is known in the art. It is desirable to formulate drugs in carriers that facilitate oral administration since this is less traumatic for subjects in need of drug treatment. A problem associated with oral delivery is that a drug has to transit the digestive tract and although some drugs are absorbed in the stomach, others are absorbed in the small and large intestine. Drug delivery vehicles are available that protect and delay the release of drugs until they reach the intestine where they are released and subsequently absorbed. In some examples the drugs are immediately released and absorbed resulting in a rapid increase in the concentration of the drug. However, a consequence of immediate release is that if a drug has undesirable side effects these can be amplified by the sudden increase in drug concentration. In an effort to control drug release drug delivery vehicles have been designed that release an active agent in a controlled sustained manner resulting in a gradual increase in drug concentration. Means to deliver drugs in a sustained manner are known in the art and include slow release polymers that are formulated with a drug to control its release.

For example, drug delivery polymers, for example hydrophilic polymers, are known in the art that allow the controlled release of therapeutic agents either by diffusion out of the polymer matrix or by erosion of the polymer or a combination thereof. Polymers are degradable or non-degradable but degradable are preferred since they degrade to smaller molecules that excreted. Examples of these polymers are cellulose based polymers. Examples of hydrophilic drug polymers include hydroxypropylmethylcellulose, hydroxypropyl cellulose, methyl cellulose, sodium carboxymethylcellulose, poly(ethylene)oxide, polymethyacrylates or polyvinyl alcohol. The polymer morphology can also affect the release properties of the encapsulated drug and typically polymer matrices can be in the form of micro/nanospheres.

Examples of polymers used to obtain sustained release of a drug are provided in WO99/22724 which describes the use of hydrophilic drug delivery polymers in the sustained release of venlafaxine an anti-depressant. JP2006335771 discloses the use of hydroxypropylmethylcellulose in the delivery of a number of medicines; similarly, WO0110443 describes the use of hydroxypropylmethylcellulose in the sustained delivery of the anti-cancer agent camptothecin. It is apparent that means to deliver drugs in a sustained pattern are known in the art.

Hypothyroidism is a condition that results from a failure of the thyroid gland to secrete a physiologically sufficient amount of thyroid hormone. There are many symptoms associated with hypothyroidism and these include fatigue, weakness, weight gain, bradycardia, cardiomyopathy, hyperlipidaemia, hair loss, cold intolerance, constipation, depression, abnormal menstrual cycles and decreased libido. There are two common causes of hypothyroidism. The first involves inflammation or autoimmunity to the thyroid gland which results in damage to the hormone secreting cells and failure of thyroid hormone secretion. A common form of thyroid inflammation results from the autoimmune disease Hashimoto's thyroiditis. A second common cause of hypothyroidism results from surgical treatment of other conditions that require removal of all or part of the thyroid gland as, for example after surgical removal of a cancerous thyroid gland. A less common cause of hypothyroidism results from secondary effects produced on a normal thyroid gland that causes a decrease in thyroid hormone secretion. For example, if the pituitary gland fails to produce enough thyroid stimulating hormone (TSH) then the result is a lack of stimulation of the thyroid to produce thyroid hormone.

A further consequence of hypothyroidism is a secondary effect by the pituitary gland to produce large amounts of TSH to stimulate the thyroid gland to produce more thyroid hormone. In this event the thyroid gland becomes enlarged to form a goiter to compensate for reduced hormone secretion.

Individuals that suffer from hypothyroidism are typically prescribed thyroxine (T4) as replacement therapy. It is well recognised that a large proportion of patients persist with specific symptoms and a failure to regain a normal sense of wellbeing despite thyroxine replacement. For example, recent studies, it is estimated that between 30 and 50% of patients reported dissatisfaction with their treatment. The thyroid gland secretes two hormones: thyroxine (T4) and triiodothyronine (T3). T3 is the more active thyroid hormone but it is secreted at lower levels and T4 may be at least, in part, converted to T3 in the circulation. It is apparent that current treatment regimes for hypothyroidism do not adequately control the condition and alternative improved treatments are needed that do not have the side effects associated with T4 treatment. It is therefore desirable to provide a treatment for hypothyroidism that provides a physiological dosage regime to entrain thyroid hormone secretion.

We disclose a formulation comprising both T4 and T3 in combination with a means to control the release of each hormone in a sustained pattern. The formulation is administered to a subject in need of treatment for hypothyroidism in accordance with the circadian profile of T4 and T3. Both T4 and T3 are released substantially simultaneously in a sustained manner. As T4 has a long half-life of 7 days this will result in constant physiological concentration of Ft4. In contrast T3 has a much shorter half-life of approx 12 hours and thus sustained release will result in a circadian profile of Ft3. If the medication is taken in the evening then drug release will result in a rise of Ft3 to peak overnight. This profile of Ft4 and Ft3 will reproduce the circadian rhythm of thyroid hormones. The problem with current formulations of thyroid hormones are that because of their immediate release and the different pharmacokinetics of T4 and T3 it is not. possible to provide physiological replacement and 25 to 50% of patients have a poor quality of life that they believe is as a consequence of inadequate thyroid replacement therapy. A tablet that combines T4 and T3 in the appropriate ratio with sustained release will allow the simulation of physiological rhythms of Ft3 and Ft4 this will have the benefit of improving quality of life, controlling TSH levels with their normal circadian rhythm and provide physiological replacement that will restore normal metabolism.

According to an aspect of the invention there is provided a medicament comprising a pharmaceutically effective amount of thyroxine and triiodothyronine and a means to release both thyroxine and triiodothyronine in a sustained release pattern when administered to a subject, preferably a human.

In a preferred embodiment of the invention at least 100 μg of thyroxine is provided in the medicament.

In a preferred embodiment of the invention at least 6 μg of triiodothyronine is provided in the medicament.

In a preferred embodiment of the invention thyroxine is provided in the medicament at 25 to 200 μg per unit dosage; preferably triiodothyronine is provided in the medicament at 1 to 20 μg per unit dosage.

In a preferred embodiment of the invention thyroxine is provided at a concentration of about 100 μg per unit dosage and triiodothyronine is provided at a concentration of about 6 μg per unit dosage.

Typically, a molar ratio of about 14:1 T4:T3, delivering around 100 μg T4 and 6 μg T3 per day is desired and the typical dose of T4 for fully hypothyroid individuals is around 1.6 μg/kg/day. As physiological replacement is based on weight, a range of tablets or equivalent dosage forms (e.g., capsules) will be required providing different quantities of T4 and T3 but at the same ratio which would be evident to the skilled person.

In a preferred embodiment of the invention said medicament comprises polymers that facilitate the release of thyroxine and triiodothyronine in a sustained pattern.

In a preferred embodiment of the invention said polymers are hydrophilic polymers.

In a preferred embodiment of the invention said hydrophilic polymers are cellulose based.

Preferably said cellulose based polymers are selected from the group consisting of: hydroxypropylmethylcellulose, hydroxypropyl cellulose, methyl cellulose, or sodium carboxymethylcellulose.

In a preferred embodiment of the invention said polymer is hydroxypropylmethylcellulose,

In an alternative preferred embodiment of the invention said polymer is starch, including pre-gelatinised starch.

In an alternative preferred embodiment of the invention said hydrophilic polymers are selected from the group consisting of polymethyacrylates and derivatives thereof (e.g., Eudragit RL and RS), polyvinyl pyrrolidone, polyvinyl alcohol, polyethelyene glycol, [poly (lactide-co-glycolide), poly(ethylene)oxide].

In a preferred embodiment of the invention said hydrophilic polymer is polymethyl methacrylate.

In an alternative preferred embodiment of the invention said polymer is non hydrophilic.

In a preferred embodiment of the invention said non hydrophilic polymer is selected from the group consisting of water insoluble ethyl derivatives (e.g., ethyl cellulose), microcrystalline cellulose (Avicel), and dicalcium phosphate.

In a preferred embodiment of the invention said medicament is a multiparticulate formulation.

In a preferred embodiment of the invention said multiparticulate formulation comprises T3 and T4 intimately mixed or co-mixed in individual microparticulate units and contained within a capsule.

In a preferred embodiment of the invention said multiparticulate formulation comprises T3 and T4 in separate multiparticulate units appropriately mixed and contained within a capsule.

Preferably said multiparticulate formulation comprises polyvinylpyrrolidone.

In a preferred embodiment of the invention said multiparticulate comprises polyvinylpyrrolidone and microcrystalline cellulose (e.g. Avicel).

In a preferred embodiment of the invention said multiparticulate comprises polyvinylpyrrolidone and dicalcium phosphate.

In a preferred embodiment of the invention said multiparticulate comprises polyvinylpyrrolidone and lactose.

In a preferred embodiment of the invention said multiparticulate comprises polyvinylpyrrolidone and a mixture of two or more of the following: microcrystalline cellulose (e.g. Avicel), dicalcium phosphate, lactose.

In a preferred embodiment of the invention polyvinylpyrrolidone is provided at between 0.5% w/w and 5% w/w; preferably 1-3% w/w.

In a preferred embodiment of the invention polyvinylpyrrolidone is provided at about 1% w/w.

Drug delivery polymers may also include excipients that can be added to a polymer drug matrix to further modify drug release, drug stability or polymer degradation kinetics or combinations thereof. For example basic salts can be added that control polymer degradation thereby altering drug release. Hydrophilic excipients can be added that accelerate drug release.

When administered, the medicament of the present invention is administered in pharmaceutically acceptable preparations. Such preparations may routinely contain pharmaceutically acceptable concentrations of salt, buffering agents, preservatives, compatible carriers and supplementary potentiating agents. The preferred route of administration is oral.

The medicament of the invention is administered in effective amounts. An “effective amount” is that amount of a medicament that alone, or together with further doses, produces the desired response. In the case of treating a particular disease the desired response is inhibiting the progression of the disease. This may involve only slowing the progression of the disease temporarily, although more preferably, it involves halting the progression of the disease permanently. This can be monitored by routine methods or can be monitored according to diagnostic methods known in the art.

Such amounts will depend, of course, on the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons. The medicaments used in the foregoing methods preferably are non-sterile, if intended for non-parenteral route and sterile if intended for parenteral administration, and contain an effective amount of thyroxine and triiodothyronine for producing the desired response in a unit of weight or volume suitable for administration to a patient.

The doses of thyroxine and triiodothyronine administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Other protocols for the administration of the medicament will be known to one of ordinary skill in the art, in which the dose amount, schedule of injections, sites of injections, mode of administration and the like vary from the foregoing. Administration of the medicament to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the medicament of the invention is applied in pharmaceutically-acceptable amounts and in pharmaceutically-acceptable compositions. The term “pharmaceutically acceptable” means physiologically or toxicologically tolerable. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The medicament may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The medicament may contain suitable buffering agents, including: acetic acid in a salt; citric acid in a salt; boric acid in a salt; and phosphoric acid in a salt. Medicaments also may contain, optionally, suitable preservatives, such as: benzalkonium chloride; chlorobutanol and parabens.

The medicament may conveniently be presented in unit dosage form and may be prepared by any of the methods well-known in the art of pharmacy. All methods include the step of bringing the active agent into association with a carrier which constitutes one or more accessory ingredients. In general, the medicament is prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

According to a further aspect of the invention there is provided the combined use of sustained release thyroxine and sustained release triiodothyronine in the manufacture of a medicament for the treatment of hypothyroidism.

In a preferred embodiment of the invention the medicament is administered to a subject wherein the administration pattern of the medicament comprises the sustained release of thyroxine and the sustained release of triiodothyronine.

In a preferred embodiment of the invention said sustained release for both thyroxine and triiodothyronine is between 4 to 10 hours.

In a preferred embodiment of the invention said medicament is administered between 18:00 h and 00:00 h.

In a preferred embodiment of the invention the administration pattern of the medicament reproduces a circadian rhythm of Ft3 in said subject.

In a preferred embodiment of the invention the administration pattern of the medicament reproduces a constant concentration of Ft4 in said subject.

In a preferred embodiment of the invention hypothyroidism results from inflammation of the thyroid gland, for example Hashimoto's thyroiditis and autoimmune hypothyroidism.

In a further preferred embodiment of the invention hypothyroidism results from surgical removal of all or part of a subject's thyroid gland.

In a further preferred embodiment of the invention there is provided a medicament for use in the treatment of the elevated levels of TSH caused by hypothyroidism and through this treatment the restoration of the normal circadian rhythm of TSH.

In a further preferred embodiment of the invention there is provided a medicament for use in the treatment of fatigue and impaired quality of life caused by hypothyroidism.

In a further preferred embodiment of the invention there is provided a medicament for use in the treatment of weight gain caused by hypothyroidism.

In a further preferred embodiment of the invention there is provided a medicament for use in the treatment of depression caused by hypothyroidism.

In a further preferred embodiment of the invention there is provided a medicament for use in the treatment of hair loss caused by hypothyroidism.

In a further preferred embodiment of the invention there is provided a medicament for use in the treatment of cardiac changes caused by hypothyroidism.

In a further preferred embodiment of the invention there is provided a medicament for use in the treatment of lipid changes caused by hypothyroidism.

In a further preferred embodiment of the invention there is provided a medicament for use in the treatment of an abnormal menstrual cycle caused by hypothyroidism.

In a further preferred embodiment of the invention there is provided a medicament for use in the treatment of decreased libido caused by hypothyroidism.

In a further preferred embodiment of the invention there is provided a medicament for use in the treatment of abnormal bone turnover and growth caused by hypothyroidism.

According to an aspect of the invention there is provided a method for the treatment of hypothyroidism comprising administering a medicament comprising an effective amount of a combined preparation of thyroxine and triiodothyronine wherein both thyroxine and triiodothyronine are released in a sustained pattern when administered to a subject.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

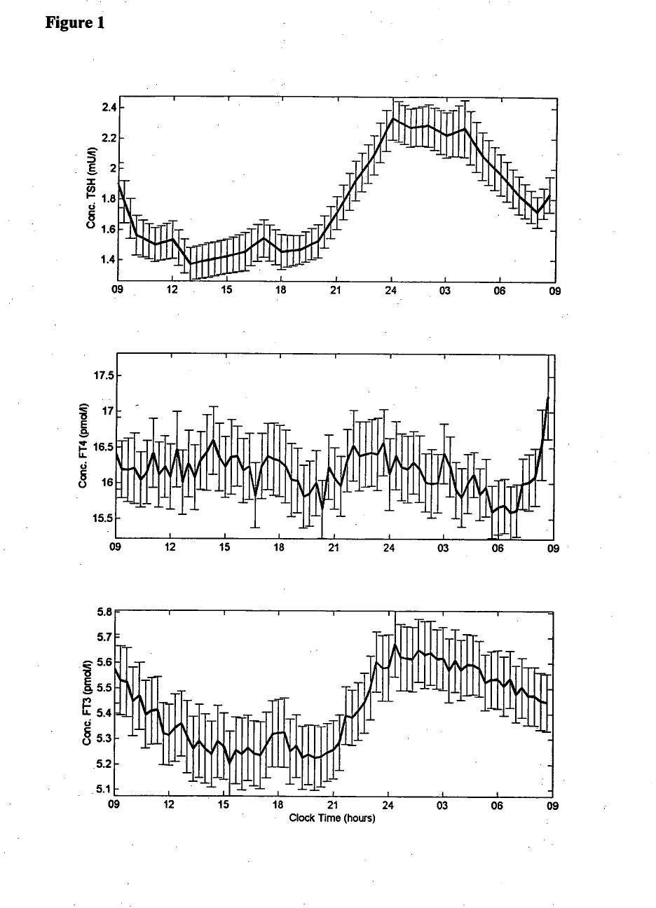

FIG. 1: illustrates the 24 hour profiles for TSH, FT4 and FT3, Mean±sem;

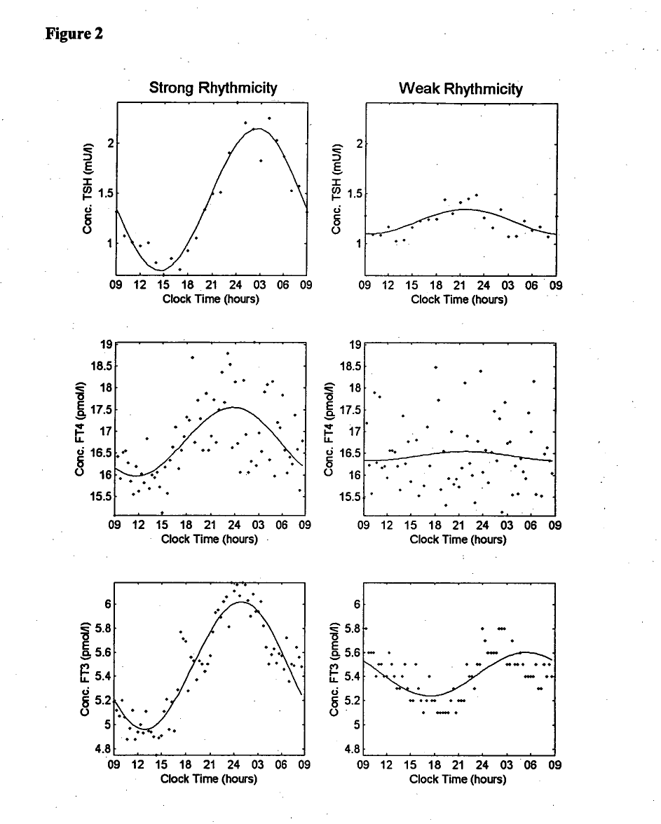

FIG. 2: illustrates representative TSH, FT4 and FT3 data from two subjects; one showing strong and the other weak rhythmicity. Estimated cosinor (solid) and raw data (dots);

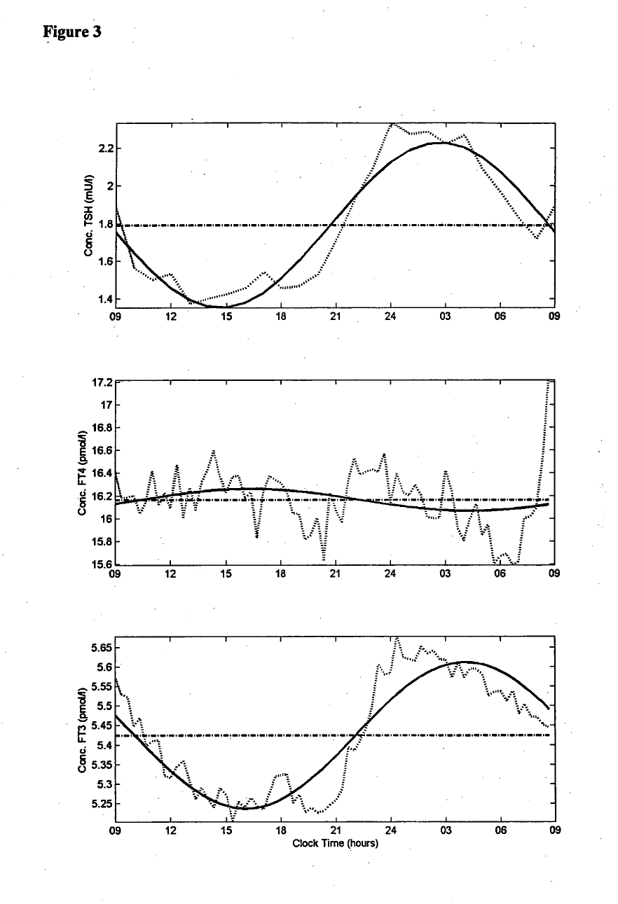

FIG. 3: illustrates mean TSH, FT4 and FT3 data (solid) with group cosinor model superimposed (dotted);

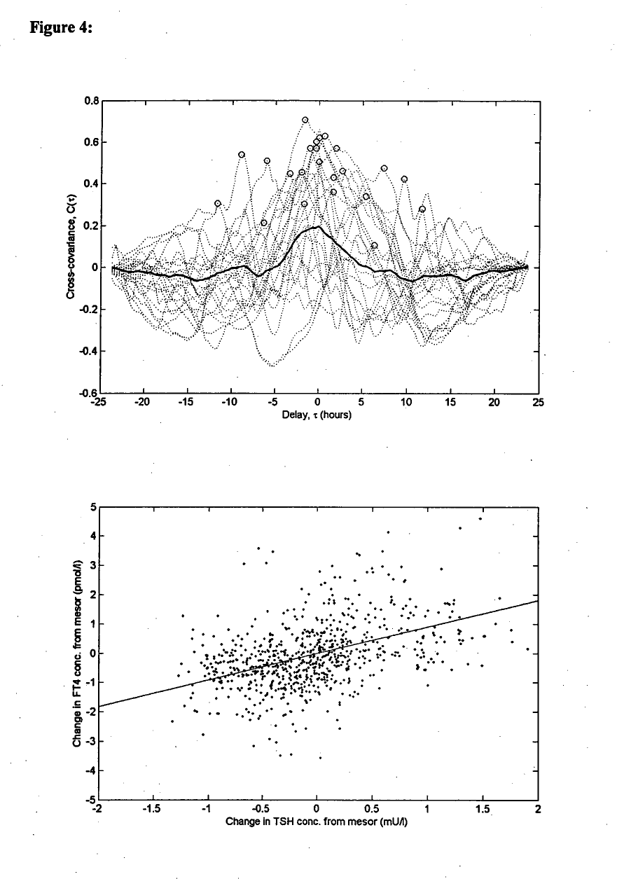

FIG. 4: illustrates Top Panel: Cross-covariance function, C(τ), of individual profiles of FT4 and TSH. The graph shows the correlation between the two profiles on the y axis for different periods of delay or advance on the x axis. If both profiles were identical both in profile and timing there would be a peak correlation of 1 at zero delay. The mean function is superimposed (solid line): a negative value of delay indicates that FT4 follows TSH. It is evident that there is negligible correlation between the FT4 and TSH profiles. Lower Panel: Scatter plot of time-shifted FT4 and TSH profiles, ρ=0.42, p<0.0001;

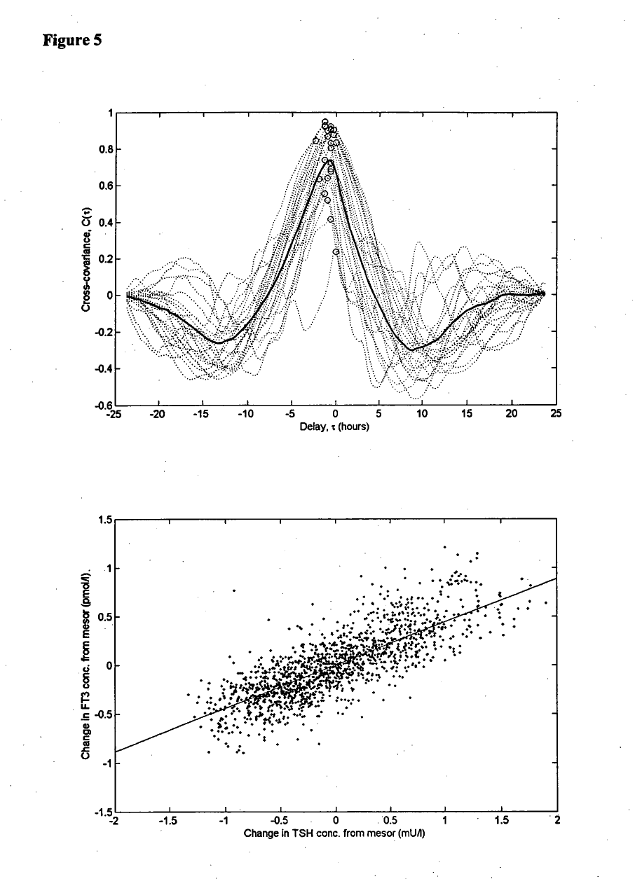

FIG. 5: illustrates Top Panel: Cross-covariance function, C(τ), of individual profiles of FT3 and TSH. The graph shows the correlation between the two profiles on the y axis for different periods of delay or advance on the x axis. If both profiles were identical both in profile and timing there would be a peak correlation of 1 at zero delay. The mean function is superimposed (solid line): a negative value of delay indicates that FT3 follows TSH. It is evident that there is considerable correlation between the FT3 and TSH profiles. Lower Panel: Scatter plot of time-shifted FT3 and TSH profiles, ρ=0.80, p<0.0001;

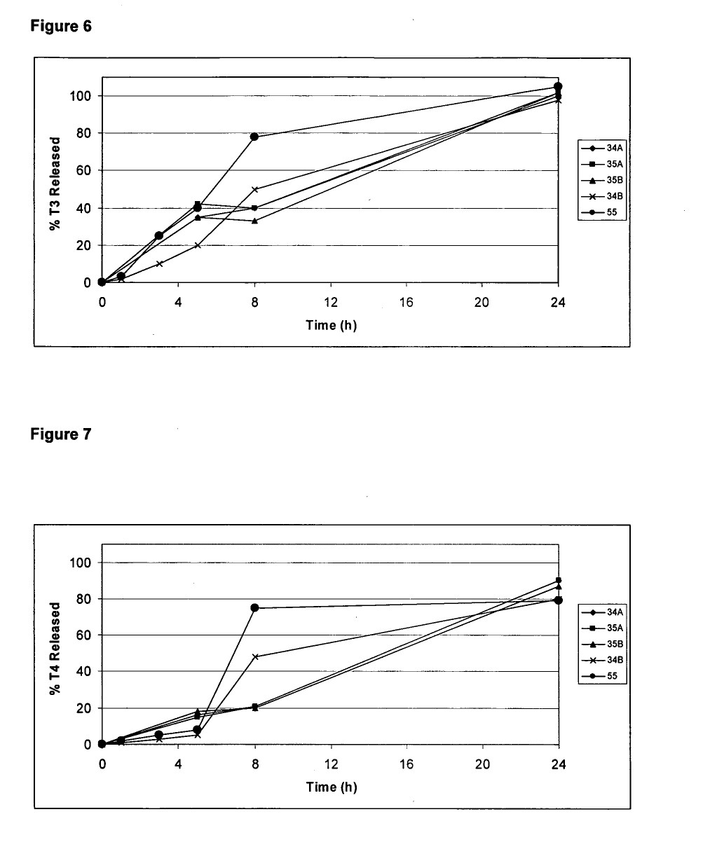

FIG. 6: illustrates mean dissolution profiles for T3 from HPMC based tablet formulations having compositions as described in Tables 1-4. The legend denotes the formulation types with the pre-fix DIU001/D;

FIG. 7: illustrates dissolution profiles for T4 from HPMC based tablet formulations having compositions as described in Tables 1-4. The legend denotes the formulation types with the pre-fix DIU001/D;

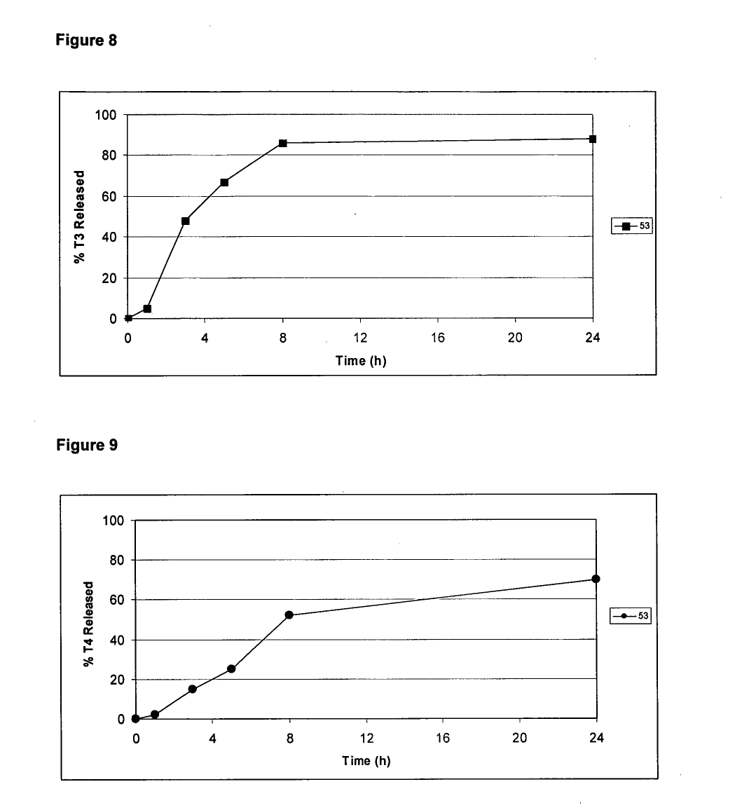

FIG. 8: illustrates dissolution profile for T3 from a multiparticulate formulation having a composition as described in Table 6. The legend denotes the formulation types with the pre-fix DIU001/D.

FIG. 9: illustrates dissolution profile for T4 from a multiparticulate formulation having a composition as described in Table 6. The legend denotes the formulation types with the pre-fix DIU001/D.

Table 12 illustrates percentage of individuals displaying significant rhythm for a range of significance levels; and

Table 13 illustrates Cosinor parameters for TSH, FT4 and FT3. Mean with 95% confidence intervals shown in parenthesis.

Materials and Methods

Matrix Tablet Formulations

The formulation composition details for HMPC and Sodium carboxymethyl cellulose (Carbopol) based tablets are as shown in Tables 1-4 and 5 respectively. These formulations contain a combination of both thyroxine (T4) and triiodothyronine (T3) in matrices which differ principally in the rate-controlling polymer type, grade and level. HPMC based tablet formulations have been prepared with two specific rate-controlling polymers viz: Hydroxypropyl methyl cellulose (HPMC—grade k4M and k15M) and Polymethyl methacrylate (Carbopol—grade 971 P). The viscosity of the HPMC polymer is dependent on the grade, k4M has an intrinsic viscosity of 4000 mPa, and k15M has an intrinsic viscosity of 15,000 mPa. Other excipients used in the formulations include: Polyvinylpyrrolidone (PVP)—grade K30, Anhydrous Dicalcium Phosphate (Emcompress), Talc, Aerosil—grade 200, and Magnesium Stearate. The functions of these excipients are as described in the Formulation Composition tables below.

Manufacturing Method

The method of manufacture for the HPMC and Carbopol matrix tablet formulations is well established in the art, and is commonly referred to as direct compression, wherein the formulation composition is blended together in a homogenous manner and compressed into a suitable tablet using a standard tablet press. A more detailed description of the process is provided below.

A. Method of Manufacture for HPMC Tablet Formulations (6 μq Triiodothyronine and 100 μq Thyroxine Per Tablet)

Excipients were sieved through a 250 μm sieve and blended using an Erweka cube mixer at a setting of 200 (29 rpm). Due to the small powder quantities involved, the actives (i.e., T3 and T4) and excipients were weighed into a 500 ml glass screw-topped bottle, which was secured to the interior wall of the mixing cube. Excipients were added to T3 and T4 actives by trituration in the following order:
1. The actives and PVP were mixed for 2 min (blend A)
2. Blend A and HPMC were mixed for 5 min (blend B)
3. Blend B and anhydrous Emcompress were mixed for 5 min (blend C)
4. Blend C and talc were mixed for 2 min (blend D)
5. Blend D and magnesium stearate were mixed for 2 min (final blend)

The resulting powder blend was compressed using a Manesty F3 tablet press with a 5.5mm (normal concave) punch set. The tablet press was adjusted to apply sufficient compression to produce a hard tablet with a smooth surface.

B. Method of Manufacture for Carbopol Tablet Formulation (6 μg Triiodothyronine and 10 μg Thyroxine Per Tablet)

Excipients were sieved through a 250 μm sieve and blended using an Erweka cube mixer at a setting of 200 (29 rpm). Due to the small powder quantities involved, the active and excipients were weighed into a 500 ml glass screw-topped bottle, which was secured to the interior wall of the mixing cube. Excipients were added to T3 and T4 actives by trituration in the following order:
1. The actives and anhydrous Emcompress were mixed for 5 min (blend A)
2. Blend A and Carbopol 971 P were mixed for 5 min (Blend B)
3. Blend B and Aerosil were mixed for 2 min (Blend C)
4. Blend C and magnesium stearate were mixed for 2 min (final blend)

The resulting powder blend was compressed using a Manesty F3 tablet press with a 5.5mm (normal concave) punch set. The tablet press was adjusted to apply sufficient compression to produce a hard tablet with a smooth surface.

Analytical Method

Dissolution data for the formulations were acquired in 500 mL 0.01 M hydrochloric acid using paddle (USP II) apparatus at 100 rpm over 24 hours. The temperature of the dissolution medium was maintained at 37° C.±0.5° C. Samples (50 mL) were taken for off-line HPLC assay after 1, 3, 5, 8 and 24 hours. The total volume in each dissolution vessel was made up to 500 mL after sampling by the addition of 50 mL 0.01M hydrochloric acid, preheated to 37° C. A correction was applied to dissolution assay data to correct for the dilution involved.

The entire 50 mL sample was pre-concentrated prior to analysis using a 500 mg C₁₈ solid phase extraction cartridge. The final extract volume was 1.0 mL, 50 μL of which was injected onto a high performance liquid chromatograph equipped with a 150 mm×4.6 mm column packed with octadecasilyl-bonded silica with a mean particle diameter of 5 μm. The column was maintained at 30° C. The mobile phase comprised 68% water containing 0.05% trifluoroacetic acid and 32% acetonitrile containing 0.05% trifluoroacetic acid at a flow rate of 1.00 mL/min. Triiodothyronine (T3) and thyroxine (T4) and were detected and quantified using an ultraviolet detector operating at 230nm. The retention times of thyroxine and triiodothyronine under these conditions were approximately 17.5 min and 8.9min respectively.

The dissolution profiles for T3 and T4 from the HPMC and Carbopol based tablet formulations are as shown in FIGS. 6 and 7 respectively. It can be noted that the level of release for T3 and T4 can be sustained over a period of up to 24 hours consistent with the expected duration of the circadian release profile in vivo. The rate of release may also be refined by the altering the polymer type, grade and level incorporated within the formulations.

TABLE 1
Formulation composition for Thyroid HPMC based tablet formulation
(DIU001/D/034A) containing low viscosity grade HPMC (k4M) at a
concentration of approximately 35%.

| | | | Proportion of |
|---|---|---|---|
| | Component | Mass per | formulation |
| Component | Function | tablet (mg) | (% w/w) |
| T₃ | Active | 0.0062 | 0.00827 |
| T₄ | Active | 0.0998 | 0.13307 |
| PVP K30 | Binder | 0.7500 | 1.00000 |
| HPMC (k4M) | Rate controlling | 34.5000 | 46.00000 |
| | polymer | | |
| Anhydrous Emcompress | Compression aid | 37.3940 | 49.85866 |
| Talc | Glidant | 1.5000 | 2.00000 |
| Magnesium Stearate | Lubricant | 0.7500 | 1.00000 |
| Total | | 75.0000 | 100.00000 |

TABLE 2
Formulation composition for Thyroid HPMC based tablet formulation
(DIU001/D/035A) containing low viscosity grade HPMC (k4M) at a
concentration of approximately 54%.

| | | | Proportion of |
|---|---|---|---|
| | Component | Mass per | formulation |
| Component | Function | tablet (mg) | (% w/w) |
| T₃ | Active | 0.0062 | 0.00827 |
| T₄ | Active | 0.0998 | 0.13307 |
| PVP K30 | Binder | 0.7500 | 1.00000 |
| HPMC (k4M) | Rate controlling | 53.6250 | 71.50000 |
| | polymer | | |
| Anhydrous Emcompress | Compression aid | 18.2690 | 24.35866 |
| Talc | Glidant | 1.5000 | 2.00000 |
| Magnesium Stearate | Lubricant | 0.7500 | 1.00000 |
| Total | | 75.0000 | 100.00000 |

TABLE 3
Formulation composition for Thyroid HPMC based tablet formulation
(DIU001/D/034B) containing high viscosity grade HPMC (k15M) at a
concentration of approximately 35%.

| | | | Proportion of |
|---|---|---|---|
| | Component | Mass per | formulation |
| Component | Function | tablet (mg) | (% w/w) |
| T₃ | Active | 0.0062 | 0.00827 |
| T₄ | Active | 0.0998 | 0.13307 |
| PVP K30 | Binder | 0.7500 | 1.00000 |
| HPMC (k15M) | Rate controlling | 34.5000 | 46.00000 |
| | polymer | | |
| Anhydrous Emcompress | Compression aid | 37.3940 | 49.85866 |
| Talc | Glidant | 1.5000 | 2.00000 |
| Magnesium Stearate | Lubricant | 0.7500 | 1.00000 |
| Total | | 75.0000 | 100.00000 |

TABLE 4
Formulation composition for Thyroid HPMC based tablet formulation
(DIU001/D/035B) containing high viscosity grade HPMC (k15M) at a
concentration of approximately 54%.

| | | | Proportion of |
|---|---|---|---|
| | Component | Mass per | formulation |
| Component | Function | tablet (mg) | (% w/w) |
| T₃ | Active | 0.0062 | 0.00827 |
| T₄ | Active | 0.0998 | 0.13307 |
| PVP K30 | Binder | 0.7500 | 1.00000 |
| HPMC (k15M) | Rate controlling | 53.625 | 71.50000 |
| | polymer | | |
| Anhydrous Emcompress | Compression aid | 18.269 | 24.35866 |
| Talc | Glidant | 1.5000 | 2.00000 |
| Magnesium Stearate | Lubricant | 0.7500 | 1.00000 |
| Total | | 75.0000 | 100.00000 |

TABLE 5
Formulation composition for Thyroid Carbopol based tablet
formulation (DIU001/D/055B) containing Carbopol grade 971P
at a concentration of 7.5%.

| | | | Proportion of |
|---|---|---|---|
| | Component | Mass per | formulation |
| Component | Function | tablet (mg) | (% w/w) |
| T₃ | Active | 0.0062 | 0.00827 |
| T₄ | Active | 0.0998 | 0.13307 |
| Carbopol 971P | Rate controlling | 7.5000 | 10.00000 |
| | polymer | | |
| Anhydrous Emcompress | Compression aid | 66.6440 | 88.85866 |
| Aerosil 200 | Glidant | 0.3750 | 0.50000 |
| Magnesium Stearate | Lubricant | 0.3750 | 0.50000 |
| Total | | 75.0000 | 100.00000 |

2. Multiparticulate Formulations

The formulation composition detail for the multiparticulate formulations (also known as spheroids) containing a combination of both triiodothyronine (T3) and thyroxine (T4) is shown in Table 6-9. The main excipient base of these formulations are a mixture of Polyvinylpyrrolidone (PVP)—grade K90, Microcrystalline cellulose (Avicel)—grade pH101, dicalcium phosphate, lactose, magnesium oxide and sodium hydroxide (at trace level). The functions of these excipients are as described in the Formulation Composition table below.

The formulation composition detail for the multiparticulate formulations containing triiodothyronine (T3) and thyroxine (T4) in individual multiparticulate units and blended together into a common dosage form to deliver a combined release profile, is shown in Table 10. The main excipient base of these formulations is a mixture of Polyvinylpyrrolidone (PVP)—grade K90, Microcrystalline cellulose (Avicel)—grade pH101, dicalcium phosphate, lactose, magnesium oxide and sodium hydroxide (at trace level). The functions of these excipients are as described in the Formulation Composition table below.

Method of Manufacture for Multiparticulate Formulations Described in Tables 6-10

A binder solution was prepared, consisting of 10% ethanol and 90% 0.01 M aqueous sodium hydroxide solution by weight. The active(s) were dissolved in the binder solution, using sonication for 10 min. The binder: Active ratio was 1:1.305.

Excipients were sieved through a 250 μm sieve and blended using an Erweka cube mixer at a setting of 200 (29 rpm). Due to the small powder quantities involved, the actives and excipients were weighed into a 500 ml glass screw-topped bottle, which was secured to the interior wall of the mixing cube. After excipient powder blend mixing was complete, the binder solution containing the actives was slowly added (over approximately 5 min) to the powder blend, and mixed using a metal spatula until uniform.

The resulting wet mass was extruded through a Caleva miniscrew extruder, using a 1.0 mm diameter die, at 80 rpm. The extrudate was spheronised using a Caleva Spheroniser 250 at 1500 rpm using a 2 mm×2 mm square cut plate for 10 min. The multiparticulates were dried in air at ambient temperature for 2 days.

Analytical Method

The dissolution employed was common to that used for the tablet formulations as detailed above.

The dissolution profiles for T3 and T4 from a representative multiparticulate formulation are, as shown in FIGS. 8 and 9 respectively. It can be noted that the level of release for T3 and T4 can be sustained over a period of up to 24 hours consistent with the expected duration of the circadian release profile in vivo.

TABLE 6
Formulation composition for T3 and T4 combined in a
multiparticulate formulation based on co-extruded PVP and Avicel
excipient (DIU001/D/053).

| | | Proportion of |
|---|---|---|
| | | formulation |
| Component | Component Function | (% w/w) |
| T₃ | Active | 0.0100 |
| T₄ | Active | 0.1660 |
| PVP (K90) | Rate-controlling polymer | 1.0000 |
| Avicel pH101 | Diluent | 88.8240 |
| Magnesium Oxide | Stabiliser | 10.0000 |
| Total | | 100.0000 |

TABLE 7
Formulation composition for T3 and T4 combined in a
multiparticulate formulation based on co-extruded PVP and dicalcium
phosphate excipient (DIU001/D/053-B).

| | | Proportion of |
|---|---|---|
| | | formulation |
| Component | Component Function | (% w/w) |
| T₃ | Active | 0.0100 |
| T₄ | Active | 0.1660 |
| PVP (K90) | Rate-controlling polymer | 1.0000 |
| Dicalcium phosphate | Diluent | 88.8240 |
| Magnesium Oxide | Stabiliser | 10.0000 |
| Total | | 100.0000 |

TABLE 8
Formulation composition for T3 and T4 combined in a multiparticulate
formulation based on co-extruded PVP and Lactose (DIU001/D/053-C).

| | | Proportion of |
|---|---|---|
| | | formulation |
| Component | Component Function | (% w/w) |
| T₃ | Active | 0.0100 |
| T₄ | Active | 0.1660 |
| PVP (K90) | Rate-controlling polymer | 1.0000 |
| Lactose | Diluent | 88.8240 |
| Magnesium Oxide | Stabiliser | 10.0000 |
| Total | | 100.0000 |

TABLE 9
Formulation composition for T3 and T4 combined in a multiparticulate
formulation based on co-extruded PVP and a mixture of Avicel,
dicalcium phosphate and lactose excipients (DIU001/D/053-D).

| | | Proportion of |
|---|---|---|
| Component | Component Function | formulation (% w/w) |
| T₃ | Active | 0.0100 |
| T₄ | Active | 0.1660 |
| PVP (K90) | Rate-controlling polymer | 1.0000 |
| Avicel pH101 | Diluent | 48.8240 |
| Dicalcium phosphate | Diluent | 20.0000 |
| Lactose | Diluent | 20.0000 |
| Magnesium Oxide | Stabiliser | 10.0000 |
| Total | | 100.0000 |

TABLE 10
Formulation composition for separate T3 and T4 multiparticulates
co-mixed into a dosage form, based on co-extruded PVP and Avicel
excipients (DIU001/D/053)

| | | Proportion of formulation |
|---|---|---|
| Component | Component Function | (% w/w) |
| T₃ | Active | 0.0100 |
| PVP (K90) | Rate-controlling polymer | 1.0000 |
| Avicel pH101 | Diluent | 88.9900 |
| Magnesium Oxide | Stabiliser | 10.0000 |
| Sub-total | | 100.0000 |
| T4 | Active | 0.1660 |
| PVP (K90) | Rate-controlling polymer | 1.0000 |
| Avicel pH101* | Diluent | 88.8340 |
| Magnesium Oxide | Stabiliser | 10.0000 |
| Total | | 100.0000 |

*Note that the Avicel diluent can be appropriately replaced or combined with dicalcium phosphate or lactose.

3. Formulation Stability

The stability of the actives (T3 and T4) within the above formulations has been evaluated via an accelerated screening technique. Samples were prepared in glass vials containing 14 mg of thyroxine, 1 mg of triiodothyronine and various excipients in the same approximate ratio as represented in the above exemplified formulations. The excipients present in each powder blend are listed in Table 7. The vials were stored at 25° C. and 50° C., and samples taken for analysis initially and after two and four weeks' storage. Samples, except those containing carbopol, were extracted in a 50:50 v/v mixture of methanol and 0.01 M sodium hydroxide. Formulation 4 (containing carbopol) was extracted initially with methanol, an aliquot filtered and then diluted with an equal volume of 0.01M sodium hydroxide. This approach was adopted to avoid problems with sample viscosity caused by the cross-linking of carbopol in alkaline solution. Samples were filtered through a PVDF membrane prior to analysis.

Sample Analysis

20 μL of each sample solution and calibration standards containing the same nominal concentrations of thyroxine and triiodothyronine were injected onto a high performance liquid chromatograph fitted with a 250 mm×4.0 mm column packed with octadecasilyl-bonded silica with a mean particle diameter of 5 μm, maintained at 30° C. The column was eluted using a linear water/acetonitrile gradient, modified with 0.05% v/v trifluoroacetic acid, from 92% water/8% acetonitrile to 100% acetonitrile over 25 min. The final gradient composition was held for 5 min, and the column was equilibrated at the starting mobile phase composition for 10 min between injections. Thyroxine, triiodothyronine and their related substances were detected using an ultraviolet detector at 230 nm. Degradation products were identified on the basis of increasing peak areas during stability storage, and quantified relative to the combined API peak area. The nominal concentrations of thyroxine and triiodothyronine in sample extracts and calibration standards were 0.10 mg/mL and 0.07 mg/mL respectively. Their retention times under these conditions were approximately 15.5 min (thyroxine) and 14.1 min (triiodothyronine).

Table 11 shows the amount of impurities generated as a function of accelerated testing. It can be noted that for all formulation composition and exposure temperature, the amount of degradation was less than 1% up to a 4-week period, illustrating that the excellent stability of T3 and T4 in the formulations.

TABLE 11
Stability data for T3 and T4 in the formulation excipient matrix.

| | | | Total | |
|---|---|---|---|---|
| | | | impurities | |
| | | | relative to | |
| Formu- | | | initial sample | |
| lation | Excipients | Condition | Initial | 4 week |
| 1 | HPMC, calcium phosphate, | 25° C. | 0.00 | <0.05 |
| | magnesium stearate, colloidal silica | 50° C. | N/A | 0.18 |
| 2 | HPMC, calcium phosphate, | 25° C. | 0.00 | <0.05 |
| | magnesium stearate, talc | 50° C. | N/A | <0.05 |
| 3 | HPMC, calcium phosphate, PVP, | 25° C. | 0.00 | 0.43 |
| | magnesium stearate, talc | 50° C. | N/A | 0.70 |
| 4 | Carbopol 971P, calcium phosphate, | 25° C. | 0.00 | <0.05 |
| | PVP, magnesium stearate, talc | 50° C. | N/A | <0.05 |
| 9 | Avicel, PVP | 25° C. | 0.00 | 0.05 |
| | | 50° C. | N/A | 0.18 |

Study Design

Subjects

Samples were used from 33 healthy individuals who were studied as controls for previously published research on pituitary hormone levels in patients who have undergone cranial irradiation (1). The healthy subjects all had normal endocrine parameters and were taking no medication. The mean (range) age was 22.8 (17.3-56.5) yrs, BMI 22.9 (16.3-28.9) kg/m², and female to male ratio 9/24.

Study Procedures

The study was initially approved by the South Manchester local research ethics committee and the additional analysis of FT4 and FT3 by the South Sheffield research ethics committee. Blood sampling at 20-min intervals was carried out between 0900 and 0840 h next morning. Three standard hospital meals were provided at 0830, 1230, and 1800 h, and physical activity was restricted to within the ward. Sera were separated and immediately frozen at −80° C.

Assays

TSH levels were measured hourly on samples from 33 subjects using a third-generation TSH assay (Heterogeneous Sandwich Magnetic Separation Assay) on the Immuno 1 System (Bayer, Pittsburgh, Pa.). The sensitivity of this method is 0.005 mU/l, with a reported normal range of 0.35-3.5 mU/l. The CV at TSH levels of 0.028 mU/l was 9.8% and at 0.5 mU/l, 1.9%. TSH levels were only measured hourly in the initial analysis and there was insufficient sample to do 20 minute sampling. FT4 and FT3 were measured on 20-min samples in 29 of the subjects (samples were not available on four subjects) using the Advia Centaur Chemiluminescence analyser (Advia, Deerfield, USA). For FT4 the sensitivity is 1.3 pmol/l, the normal range 10.3-21.9 pmol/l, and the CV 6.6% at 6.1 pmol/l and 3.0% at 13.9 pmol/l. For FT3 the sensitivity is 0.3 pmol/l, the normal range 3.5-6.5 pmol/l, and the CV, 4.0% at 2.9 pmol/l and 2.9% at 6.6 pmol/l.

Statistical Analysis

Linear interpolation was applied to accommodate the small number of missing values (<0.5% overall, zero in TSH) all of which were distinct, and verified by eye.

Single cosinor models of the form:

$$\begin{matrix}z ( t ) = M + A cos ( 2 π t / T + ϕ ) + e ( t ) \\ = M + γ cos ( 2 π t / T ) + β sin ( 2 π t / T ) + e ( t )\end{matrix}$$

were used to model the variation in measured hormone concentration as a function of time, t (hours). z(t), e(t) represent the measured concentration and the error between the cosinor model and the measurement, respectively. The parameter M represents the mesor (value about which the variation occurs), A, the amplitude (distance from mesor to peak) and φ (radians), the acrophase (the time of occurrence of the peak equals φT/2π). T is the period, chosen here as 24. For a fixed value of T and known values of t, simple re-arrangement of the model using trigonometric identities gives a linear model in the coefficients, M, γ, β, that can be fitted using conventional least-squares methods (2). Individual single cosinor models were fitted for each subject using least-squares and the hypothesis that the data are better explained by the null hypothesis, H0: a constant value (mesor) than, H1: a single sinusoid with 24 hour period and mean value equal to the mesor, was tested via a likelihood ratio, F, test: reject H0 for large values of F-ratio. For FT3, FT4 an F-ratio, F_{2,69} (0.95)=3.13 was considered significant while for TSH, it was F_{2,22} (0.95)=3.44.

A group cosinor model was computed by averaging the coefficients from the individual fits and the same null hypothesis was tested via the multivariate generalisation of the likelihood ratio—the modified Wilkes' lambda statistic—which can be shown to be well-approximated by the Chi-square distribution with appropriate degrees of freedom for the circumstances obtaining here (relatively large sample, few coefficients). For FT3, FT4 a Chi-square statistic, χ₅₈²(0.95)=76.8 was deemed significant, while for TSH, it was χ₆₆²(0.95)=86.0.

To look for similarities between pairs of hormone signals (TSH and FT4; TSH and FT3), the hormone profiles were shifted to maximise the correlation between the two signals by first identifying the peak value in the cross-covariogram and then re-aligning the two signals by the corresponding interval. To do this, the TSH profiles were first re-sampled on a 20 minute interval via linear interpolation. Again all records were inspected visually to ensure reasonable intersample behaviour. Profiles with time-shift exceeding 12 hours were omitted and therefore data from only 24 are presented. A scatter-plot of the remaining time-adjusted samples was then analysed for correlation (Pearson coefficient).

Cosinor Analysis of Individual Hormone Profiles

The mean profiles for TSH, FT4 and FT3 during the 24-hours are shown in FIG. 1. It should be noted that in computing these averages, no account was taken of that fact that individuals peak at different times and so a degree of smoothing should be expected. Nonetheless, visual inspection of the traces strongly supported the existence of a circadian rhythm for TSH and FT3 but not for FT4. Based on this observation we tested the hormone data for a periodic signal using single cosinor analysis and an assumption of a 24 hour period. Individual data from subjects displaying either strong or weak rhythmicity are shown in FIG. 2. Table 1 summarizes the percentage of subjects for which rejection of the null hypothesis, a straight line is better than a sinusoid, was demonstrated. It is evident that a very high proportion of subjects displayed rhythmicity in TSH and FT3; between 86-100% at p<0.05 and 76% at p<0.001, whereas FT4 achieves only 76% at p<0.05 and 46% at p<0.001.

Group Cosinor Analysis

A single cosinor model for the group for each hormone was constructed by averaging the coefficients, M,γ,β, across the group (group amplitude and acrophase are then easily computed) (2). Rejection of H0 is indicated for all three hormones, p<0.001, suggesting that the group data are well supported by the adoption of a single cosinor model. FIG. 3 shows the mean of the raw data for each hormone with the group cosinor prediction superimposed. TSH and FT3 exhibited a close fit with their group means, whereas this was less evident for FT4. Table 2 summarizes the values of the cosinor parameters for each of the hormones. TSH exhibited the greatest amplitude and the change in hormone level from nadir to peak was 0.88 mU/l; which represents a percentage change of 49.4% from the mesor value. For FT3 the nadir to peak change was 6.9% of the mesor and for FT4, 1.2%. For TSH acrophase occurred at a clock time of 0240 h and for FT3, approximately 90 minutes later at 0404 h. The model also predicts that TSH hormone levels remained above the mesor between 2020 h and 0820 h; FT3 from 2200 h to 1000 h.

Correlation of Individual Hormones

The cross-covariograms and scatter-plots for TSH and FT4 and FT3 are shown in FIGS. 4 and 5, respectively. The cross-covariograms for individual subjects suggests a close relationship between TSH and FT3 with 20 of 24 subjects showing peak correlation at between −0.5 and −2.5 hours suggesting FT3 lags TSH by these amounts. There was a strong correlation between the time-adjusted FT3 and TSH levels (p=0.80, p<0.0001). In contrast, the cross-covariogram showed no strong temporal relationship between FT4 and TSH with the peaks being spread quite uniformly. The computed group delay was zero. The scatter-plot revealed a weak correlation between FT4 and TSH (p=0.42, p<0.0001).

### REFERENCES

Darzy K H, Shalet S M 2005 Circadian and stimulated thyrotropin secretion in cranially irradiated adult cancer survivors. J Clin Endocrinol Metab 90:6490-6497
Nelson W, Tong Y L, Lee J K, Halberg F 1979 Methods for cosinor-rhythmometry. Chronobiologia 6:305-323

TABLE 12

| | percentage | | |
|---|---|---|---|
| | displaying | | |
| significance | significant rhythm | | |
| level (p) | FT3 | FT4 | TSH |
| 0.050 | 86 | 76 | 100 |
| 0.010 | 86 | 66 | 91 |
| 0.005 | 82 | 55 | 85 |
| 0.001 | 76 | 46 | 76 |

TABLE 13

| | Mesor | Amplitude | Acrophase (hours) |
|---|---|---|---|
| TSH | 1.78 (1.64-1.93) | 0.439 (0.174-0.703) | 0240 (0222-0258) |
| FT4 | 16.2 (13.5-18.8) | 0.097 (−5.81-6.01) | 1620 (1521-1719) |
| FT3 | 5.42 (5.34-5.51) | 0.187 (0.002-0.372) | 0404 (0340-0428) |

1. A medicament comprising a pharmaceutically effective amount of thyroxine and triiodothyronine and a means to release both thyroxine and triiodothyronine in a sustained release pattern when administered to a subject.

2. The medicament according to claim 1, wherein the medicament comprises at least 100 μg of thyroxine.

3. The medicament according to claim 1, wherein the medicament comprises at least 6 μg triiodothyronine.

4. The medicament according to claim 1, wherein the medicament comprises thyroxine at about 25 to 200 μg per unit dose.

5. The medicament according to claim 1, wherein the medicament comprises triiodothyronine at about 1 to 20 μg per unit dose.

6. The medicament according to claim 1, wherein the medicament comprises thyroxine at about 100 μg and triiodothyronine at about 6 μg per unit dose.

7. The medicament according to claim 1, wherein said medicament comprises polymers that facilitate the release of thyroxine and triiodothyronine in a sustained pattern.

8. The medicament according to claim 7, wherein said polymers are hydrophilic polymers.

9. The medicament according to claim 8, wherein said hydrophilic polymers are cellulose based.

10. The medicament according to claim 9, wherein said hydrophilic polymers are selected from the group consisting of:
hydroxypropylmethylcellulose, hydroxypropyl cellulose, methyl cellulose, sodium carboxymethylcellulose poly(ethylene)oxide, polymethyacrylates, and polyvinyl alcohol.

11. The medicament according to claim 10, wherein said hydrophilic polymer is hydroxypropylmethylcellulose.

12. The medicament according to claim 10, wherein said hydrophilic polymer is polymethyl methacrylate.

13. The medicament according to claim 7, wherein said polymer is non-hydrophilic.

14. The medicament according to claim 13, wherein said non hydrophilic polymer is selected from the group consisting of a water insoluble ethyl derivative, microcrystalline cellulose, and dicalcium phosphate.

15. The medicament according to claim 7, wherein said medicament is a multiparticulate formulation.

16. The medicament according to claim 15, wherein said multiparticulate formulation comprises polyvinylpyrrolidone.

17. The medicament according to claim 16, wherein said multiparticulate formulation comprises polyvinylpyrrolidone and microcrystalline cellulose.

18. The medicament according to claim 16, wherein said multiparticulate formulation comprises polyvinylpyrrolidone and dicalcium phosphate.

19. The medicament according to claim 16, wherein said multiparticulate formulation comprises polyvinylpyrrolidone and lactose.

20. The medicament according to claim 16, wherein said multiparticulate formulation comprises polyvinylpyrrolidone and at least one or all of the following: microcrystalline cellulose, dicalcium phosphate, or lactose.

21. The medicament according to claim 15, wherein said multiparticulate formulation comprises thyroxine and triiodothyronine combined in individual multiparticulate units within a capsule dosage form.

22. The medicament according to claim 15, wherein said multiparticulate formulation comprises thyroxine and triiodothyronine in separate multiparticulate units combined together within a capsule dosage form.

23. The medicament according to claim 16, wherein the medicament comprises polyvinylpyrrolidone at between 0.5% w/w and 5% w/w.

24. The medicament according to claim 23, wherein polyvinylpyrrolidone is provided between 1-3% w/w.

25. The medicament according to claim 24, wherein polyvinylpyrrolidone is provided at about 1% w/w.

26.-45. (canceled)

46. A method for the treatment of hypothyroidism comprising administering to a subject a medicament comprising an effective amount of a combined preparation of thyroxine and triiodothyronine wherein both thyroxine and triiodothyronine are released in a sustained pattern when administered to the subject.

47. The method according to claim 46, wherein said medicament is administered to the subject between 18:00 h and 00:00 h.

48. The method according to claim 46, wherein administering the medicament comprises the use of an administration pattern that reproduces a circadian rhythm of Ft3 in said subject.

49. The method according to claim 46, wherein administering the medicament comprises the use of an administration pattern that reproduces a constant concentration of Ft4 in said subject.
